(19) United States
(12) Patent Application Publication
(10) Pub. No.: US 2008/0058685 A1
(43) Pub. Date: Mar. 6, 2008

(54) Electronic Pulse Massage Apparatus Capable of Measuring Temperature

(76) Inventor: Chin-Chih Hsieh, Yunghe City (TW)

Correspondence Address: HDSL, 4331 STEVENS BATTLE LANE, FAIRFAX, VA, 22033, US

(21) Appl. No.: 11/469,806

(22) Filed: Sep. 1, 2006

(51) Int. Cl. A61H 1/00; A61H 1/02; A61N 1/00

(52) U.S. Cl. 601/20; 601/21; 607/46

(57) ABSTRACT

An electronic pulse massage apparatus is capable of measuring temperature and includes a main body, a display screen for showing status of the apparatus and operation buttons for start/stop and for increasing/decreasing pulse amplitude. The electronic pulse massage apparatus comprises a temperature sensor arranged on the main body and can be placed near human body to measure temperature thereof. The sensed temperature is display on the display screen. The main body further includes connection jack for connecting to extended temperature sensor and connection jack for connecting to extended pulse-outputting pad. Therefore, user can perform temperature measurement and pulsing massage for remote portion of user.

### BACKGROUND OF THE INVENTION

1. Field of the Invention

The present invention relates to an electronic pulse massage apparatus capable of measuring temperature, especially to an electronic pulse massage apparatus capable of measuring temperature and composed of a temperature sensor.

2. Description of Prior Art

Research shows that electronic-pulse massage can perform massage for human body based on Chinese medical theory. The acupuncture positions of human body are firstly found according to acupuncture map and then pulse for simulating press, hit, knead and rub motion is applied for massage. Massage can promote blood circulation and reduce muscle pain caused by excessive exercise and fatigue. The stimulus to the human acupuncture can simulate moxibustion function and facilitate lymph circulation, remove poison and remove excessive fat.

Taiwan Gazette No. M280818 filed by the Applicant disclosed a multiple-function electronic pulse massage apparatus. A pulse generator generates an electronic pulse and the electronic pulse is sent to a pad through mode control unit. The pad is in contact with human body to provide a massage function. In normal operation, the electronic pulse massage apparatus enhances user physiology condition. The human body portion subjected to massage has higher temperature due to excitation.

The health condition of human can be preliminarily judged by certain parameters such as blood pressure or body temperature. It is also benefic to provide an electronic pulse massage apparatus integrated with physiology condition measurement function.

### SUMMARY OF THE INVENTION

The present invention is to provide an electronic pulse massage apparatus integrated with temperature sensor to perform massage function and measure physiology parameters for user.

The present invention is to provide an electronic pulse massage apparatus with connection jacks for extended temperature sensor and extended pulse-outputting pad. The extended temperature sensor and the extended pulse-outputting pad can help user to measure temperature and perform massage to body portion difficult to reach. The extended temperature sensor and the extended pulse-outputting pad can also be bent to reach the human body portion

The present invention is to provide an electronic pulse massage apparatus with temperature sensor to measure body temperature of user. The output pulse is determined according to the sensed temperature to achieve optimal massage effect.

Accordingly, the present invention provides an electronic pulse massage apparatus adapted for measuring temperature. The electronic pulse massage apparatus includes a main body, a display screen for showing status of the apparatus and operation buttons for start/stop and for increasing/decreasing pulse amplitude. The electronic pulse massage apparatus comprises a temperature sensor arranged on the main body and can be placed near human body to measure temperature thereof. The sensed temperature is display on the display screen. The main body further includes connection jack for connecting to extended temperature sensor and connection jack for connecting to extended pulse-outputting pad. Therefore, user can perform temperature measurement and pulsing massage for remote portion of user.

Moreover, the main body further includes two connection jacks for connecting to an extended temperature sensor and a pulse-outputting pad to provide temperature measurement and pulsing massage to remote portion of human body.

### BRIEF DESCRIPTION OF DRAWING

The features of the invention believed to be novel are set forth with particularity in the appended claims. The invention itself however may be best understood by reference to the following detailed description of the invention, which describes certain exemplary embodiments of the invention, taken in conjunction with the accompanying drawings in which:

FIG. 1 shows a perspective view of the present invention.

FIG. 2 shows another perspective view of the present invention.

FIG. 3 shows that a temperature sensor is connected to one connection jack.

FIG. 4 shows that two connection jacks of the electronic pulse massage apparatus are used.

FIG. 5 shows the electronic pulse massage apparatus according to another preferred embodiment of the present invention.

### DETAILED DESCRIPTION OF THE INVENTION

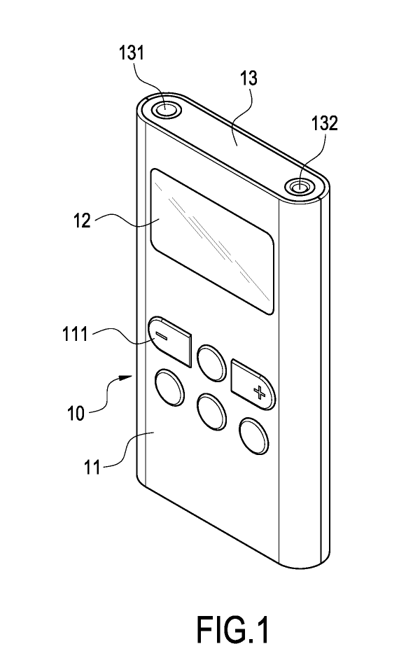

FIG. 1 shows a perspective view of the present invention. The electronic pulse massage apparatus according to the present invention comprises a main body 10, a display panel 11 with a plurality of operation buttons 111, and a display screen 12. The operation buttons 111 control functions of the electronic pulse massage apparatus such as power on, power off, amplitude level of pulse output or other customized functions. The display screen 12 displays operation status and adjustment conditions of the operation buttons 111. The upper portion 13 of the main body 10 comprises two connection jacks 131 and 132, where signals from the main body can be input and output through the connection jacks 131 and 132.

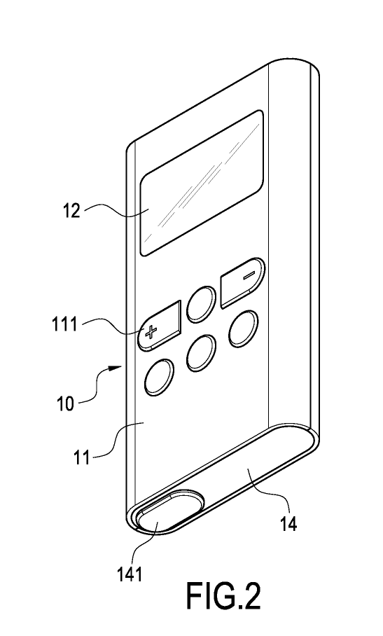

FIG. 2 shows another perspective view of the present invention. A temperature sensor 141 is provided at lower portion of the main body 10 and can use high-precision thermistor for temperature measurement. The electronic pulse massage apparatus of the present invention further comprises a sensor chip or an ADC (analog to digital converter) circuit to obtain precise voltage difference for temperature calculation. When the temperature sensor 141 is attached to a portion to be measured temperature such as human skin, the temperature sensor 141 sends a sensed temperature to the electronic pulse massage apparatus according to the present invention. Within a short time (such as several seconds), the electronic pulse massage apparatus displays the sensed temperature on the display screen 12. The arrangement position of the temperature sensor 141 in above preferred embodiment is just an exemplary example and can be placed on other place of the main body 10.

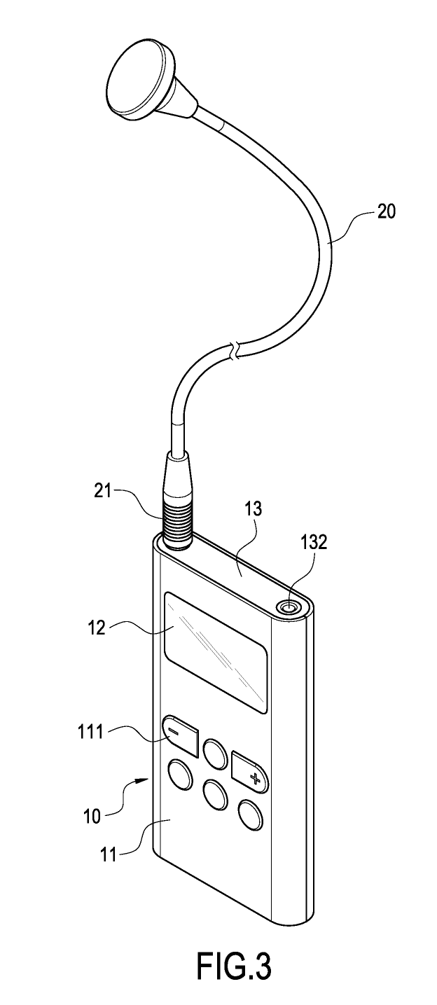

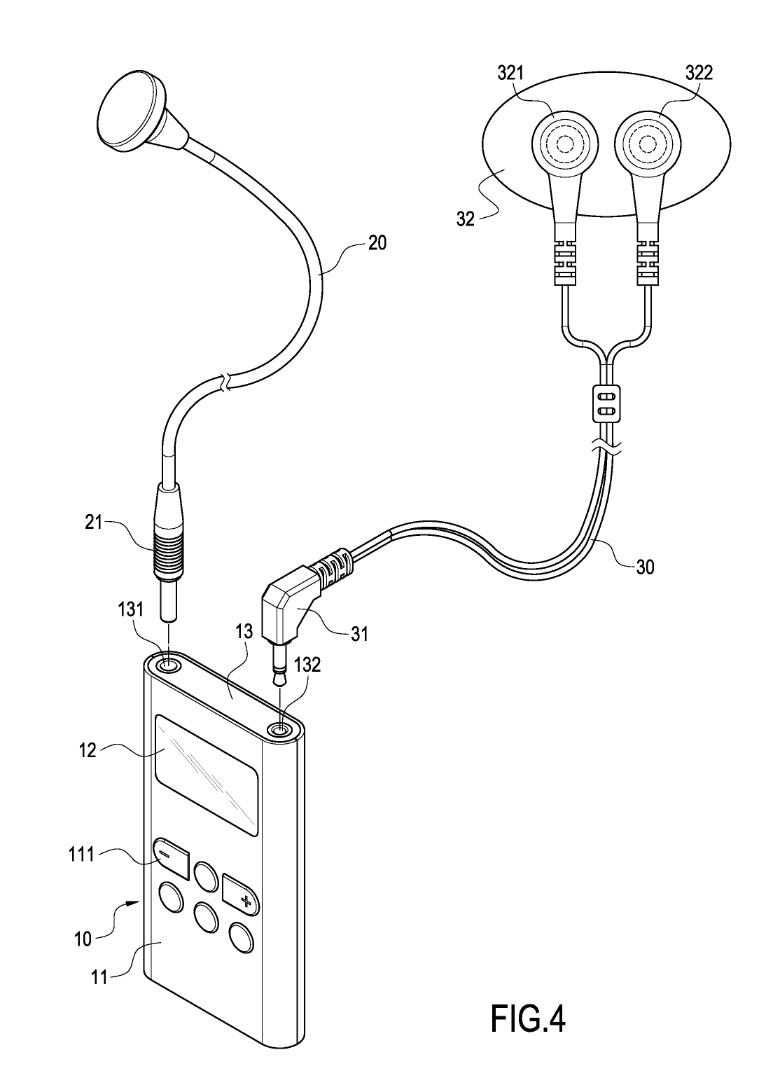

FIG. 3 shows that a temperature sensor is connected to one connection jack, where the connection end 21 of the extended temperature sensor 20 is connected with a connection jack 131 on upper portion 13 of the main body 10. Therefore, the measurement can be extended to portion difficult to reach by user such as back of user or other remote portion. FIG. 4 shows that two connection jacks 131 and 132 of the electronic pulse massage apparatus are used, where the connection end 21 of the extended temperature sensor 20 and the connection end 31 of the extended pulse-outputting pad 30 are connected to the two connection jacks 131 and 132, respectively. Therefore, the massage function and temperature measurement can be extended to portion difficult to reach by user.

The pulse-outputting pad 32 comprises two electrodes 321 and 322, and the operations thereof are controlled by operating the operation buttons 111 on the display screen 12. The control status can be manifested by the display on the display screen 12, or the output parameters of the temperature sensors 141 and 20. A processor (not shown) in the electronic pulse massage apparatus generates pulse signals based on the parameters for controlling the pulse-outputting pad 32.

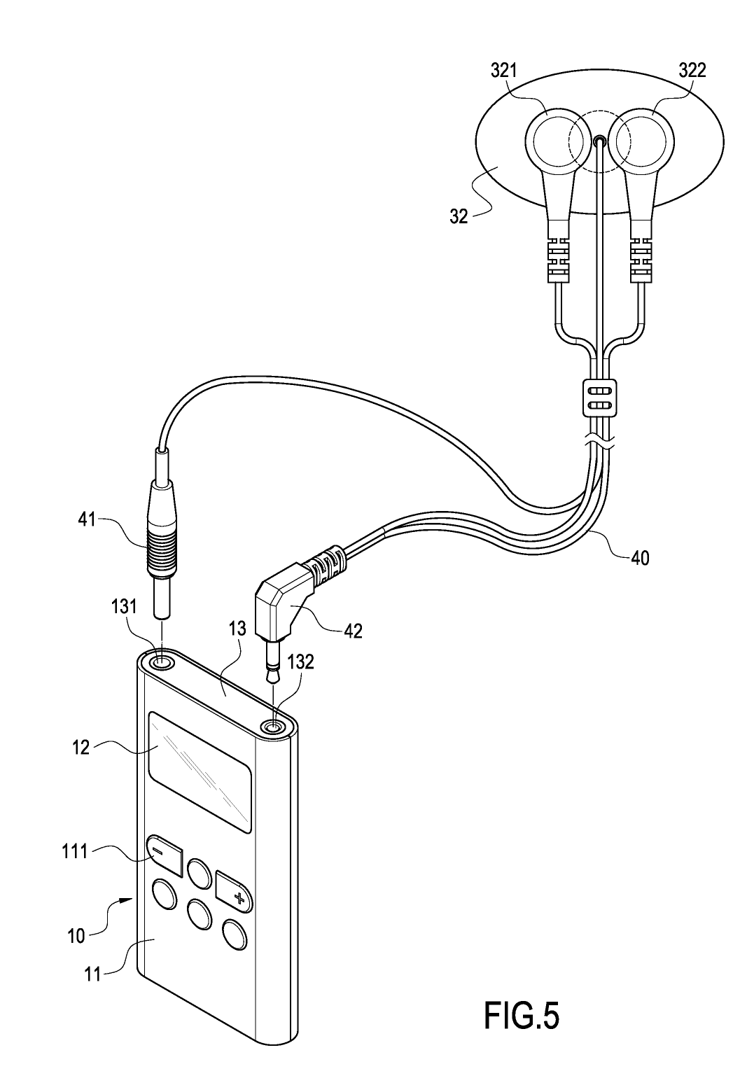

FIG. 5 shows the electronic pulse massage apparatus according to another preferred embodiment of the present invention. The above-mentioned extended temperature sensor and extended pulse-outputting pad are integrated into a temperature-sensing and pulse-outputting pad 40. The connection ends 41 and 42 are connected to different connection jacks 131 and 132. Therefore, the electronic pulse massage apparatus according to the present invention can perform temperature measurement and pulsing massage sequentially or simultaneously. Moreover, the connection ends 41 and 42 are isolated to prevent interference therebetween.

Although the present invention has been described with reference to the preferred embodiment thereof, it will be understood that the invention is not limited to the details thereof. Various substitutions and modifications have suggested in the foregoing description, and other will occur to those of ordinary skill in the art. Therefore, all such substitutions and modifications are intended to be embraced within the scope of the invention as defined in the appended claims.

What is claimed is:

1. An electronic pulse massage apparatus capable of measuring temperature, comprising:
a main body comprising an operation panel with a plurality of operation buttons, the main body comprising two connection jacks for receiving temperature signals and pulse signals;
a display screen arranged on the panel;
a temperature sensor arranged on the main body; and
an extended temperature sensor and an extended pulse-outputting pad, each with a connection end connected to corresponding one of the two connection jacks, whereby temperature measurement and pulsing massage can provide to user in a remote manner.

2. The electronic pulse massage apparatus capable of measuring temperature as in claim 1, wherein the operation buttons comprises start/stop button and adjustment buttons for increasing/decreasing pulse amplitude.

3. The electronic pulse massage apparatus capable of measuring temperature as in claim 1, wherein the body comprises upper portion and lower portion and the two connection jacks are arranged on the upper portion.

4. The electronic pulse massage apparatus capable of measuring temperature as in claim 3, wherein the temperature sensor is arranged at the lower portion.

5. The electronic pulse massage apparatus capable of measuring temperature as in claim 1, wherein the body comprises upper portion and lower portion and the temperature sensor is arranged at the lower portion.
